(19) United States
(12) Patent Application Publication
(10) Pub. No.: US 2016/0129280 A1
(43) Pub. Date: May 12, 2016

(54) LIGHT EXPOSURE REGULATING SYSTEMS AND METHODS

(71) Applicant: Verilux, Inc., Waitsfield, VT (US)

(72) Inventor: Ryan J. Douglas, Stillwater, MN (US)

(21) Appl. No.: 14/674,443

(22) Filed: Mar. 31, 2015

Related U.S. Application Data
(60) Provisional application No. 62/078,191, filed on Nov. 11, 2014.

(51) Int. Cl. A61N 5/06

(52) U.S. Cl. CPC A61N 5/0618; A61N 2005/0626; A61N 2005/0654; A61N 2005/0663

(57) ABSTRACT

Systems and methods comprising a light therapy luminaire adjustable to change a color temperature of light from the luminaire without changing the light to a non-white color; and software applications for receiving light exposure data to set a target color temperature for the luminaire. Calibration, sensors, software, and control processes for luminaires are taught.

### CROSS REFERENCE TO RELATED APPLICATIONS

This patent application claims priority to U.S. Provisional Application No. 62/078,191, Light Exposure Regulating Systems, filed Nov. 11, 2014 which is hereby incorporated by reference herein.

### TECHNICAL FIELD

The technical field relates to control of luminaires and emotional health and wellness, for instance, with data regarding a user's measured or calculated exposure to light being applied to determine how to control luminaires or other exposure to light.

### BACKGROUND

Light therapy luminaires are known for their usefulness in treating various conditions, including seasonal affective disorder.

### SUMMARY

Light can have large effects on health and emotional health. The quality and quantity of light exposure that is suitable for a person, and the need to adjust such exposure, varies from person to person and is affected by the region where a person lives, their lifestyle, physiology and other factors. But most people have little idea of how much light they are exposed to at various times in the day, the impact to their biological functions based on the light exposure and how to adjust their light exposure to improve performance and wellbeing. Even persons that actively use light therapy luminaires or the like have little guidance or help in adjusting their circumstances to best suit them and their desired result.

One solution is to use a software program to assist a user in regulating exposure to light quality/quantity. The term software program, also referred to as a program, software, a software application, an application, or an app, is broad and encompasses the means necessary to operate the software, such as a processor, as well encompassing other executable programming such as firmware, custom integrated circuits, cloud-based applications, and the like. The program can capture various data and integrate it to advise and/or control a user's light exposure both by directing the user to opportunities for more natural light exposure and by dynamically adjusting therapeutic and ambient lighting in the users' environment. Predetermined criteria can be used as a basis for the advice or control, and data collected from, and by the user can be applied to refine or create the criteria. Sensors can be worn by the user to collect data and user activities and locations can be tracked to calculate total light exposure. And the users can report data about their activities, light exposure, and their emotional wellness and/or provide user life data.

Furthermore, the light therapy arts conventionally have based recommended therapies based on light intensity, duration of exposure to light, and certain ranges of light wavelengths (a range of coloration). But what is not conventionally understood is that there is another significant factor: light color temperature. In fact, adjusting the color temperature can have a significant effect on the success or failure of a treatment. Biological factors that are not involved in vision can be important in light therapy. For instance, some light receptors are known to be stimulated by light without playing a role in vision. Without being bound to a specific theory, it is believed that an effective color temperature can be created for each user to provide effective light-based stimulation. In part, it is believed that creating a suitable color temperature allows the user to receive a higher retinal dose of light than would otherwise be tolerable. Therefore the intensity and size of the light source in a luminaire can be reduced to improve comfort and compliance without sacrificing dosage. Certain embodiments of the invention are directed to controlling light color temperature for light treatment purposes.

### BRIEF DESCRIPTION OF THE DRAWINGS

FIG. 1 Depicts a system that has a wearable light sensor and a memory/processor for control of one or more general purpose and/or light therapy luminaires.

FIG. 2 Depicts a light exposure regulating system comprising a wearable light sensing device, a memory, a processor, and with the memory configured to receive light sensor data from the light sensor, and the processor accessing the memory to process the light sensor data to provide advice about a color temperature or other output to a user of the light sensing device.

FIG. 3 Depicts a light exposure regulating system comprising a processor that applies user-self-reported data. For instance, data comprising a self-rating of emotional wellness and/or user tracking data, with the program providing an output based on the user-provided datum. The output may be in regards to color temperature, advice, a report, control of a device, and so forth.

FIG. 4 Depicts a light exposure regulating system comprising a software application configured to receive user self-reported data (e.g., data comprising a self-rating of emotional wellness) and/or user tracking data to provide automated direct control of a setting of a luminaire and/or advice for a user to control a setting of a luminaire with regards to color temperature or other light values.

FIG. 5 Depicts a light exposure regulating system comprising a processor that uses self-reported data comprising a user self-rating and/or user tracking data to provide an output of advice with regards to color temperature or other factors, based on the user self-reported data, with the processor changing the advice based on ongoing user self-reported data.

FIG. 6 Depicts a user calibrating a light therapy device.

FIG. 7 Depicts a luminaire with a set of lenses comprising a plurality of lenses.

### DETAILED DESCRIPTION

An embodiment of the invention is directed to products and methods for data-driven control of factors that include color temperature and/or recommendations for control of factors that include color temperature. A user provides data regarding exposure to light directly or indirectly to a software processor that assists the user in managing factors that include color temperature. A user may, for instance, estimate one or more of these factors and directly enter them into a software program. Or a sensor may be used that interfaces with the software to provide pertinent data. User-outcome self-rating data, collected over time, may be used to determine a setting for one or more of the factors; for instance, historical self-ratings of emotional well-being may indicate helpful patterns exposure to light, to particular light sources, to light with certain color temperature, or other factors that, in turn, can be recommended to the user in real-time. Other embodiments are directed to using a sensor and a luminaire with a changeable color temperature to establish a desired color temperature for individual users and for a particular luminaire.

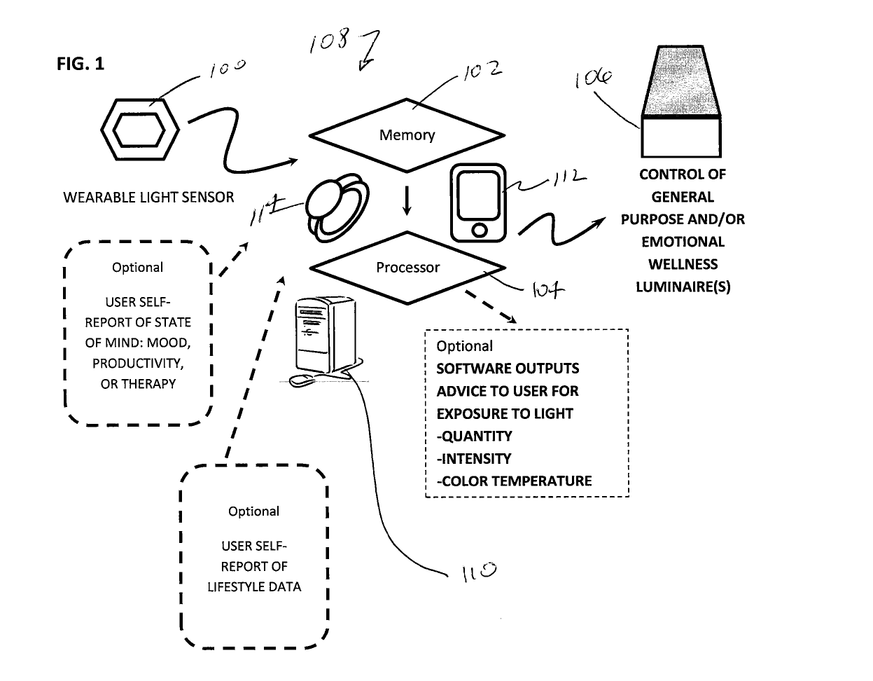

The embodiment of FIG. 1 is a system that has wearable light sensor 100, memory 102, and processor 104 for control of one or more general purpose and/or light therapy luminaires 106. The system 108 may incorporate user self-reported data and provide various outputs, such as advice to users, e.g., about exposure to light or light color temperatures. Various combination processor/memory devices are depicted, such as general purpose computer 110, mobile device (cellular telephone) 112, or wrist-worn computing device 114.

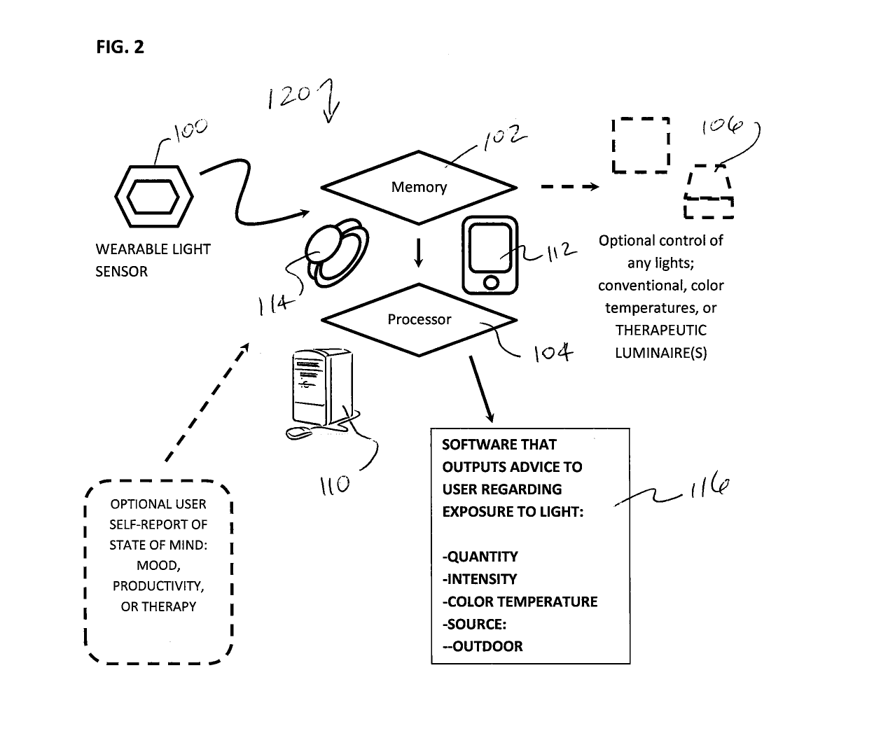

FIG. 2 depicts a light exposure regulating system 120 comprising wearable light sensing device 100, memory 102, processor 104, and with the memory configured to receive light sensor data from light sensor 100, and processor 104 accessing memory 102 to process the light sensor data to provide advice 116 about a color temperature or other output to a user of the light sensing device. The direct or indirect control of luminaires is optional in system 120.

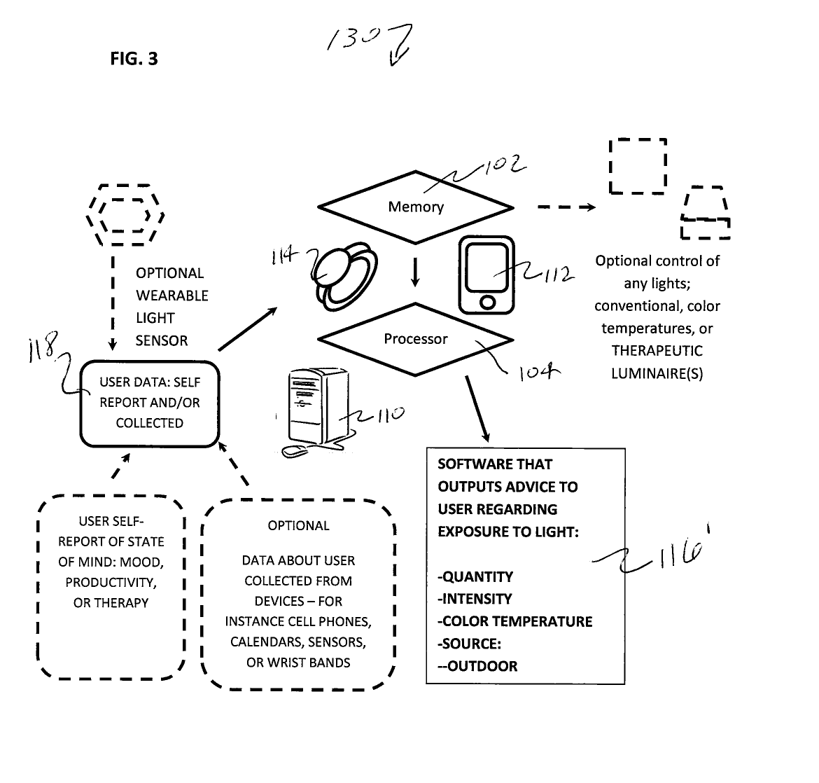

FIG. 3 depicts a light exposure regulating system 130 comprising processor 104 that applies user-self-reported data 118. For instance, data comprising a self-rating of emotional wellness and/or user tracking data may be collected, with a program operating on processor 104 providing an output 116′ based on user-provided data 118. Output 116′ may be in regards to color temperature, advice, a report, control of a device, and so forth.

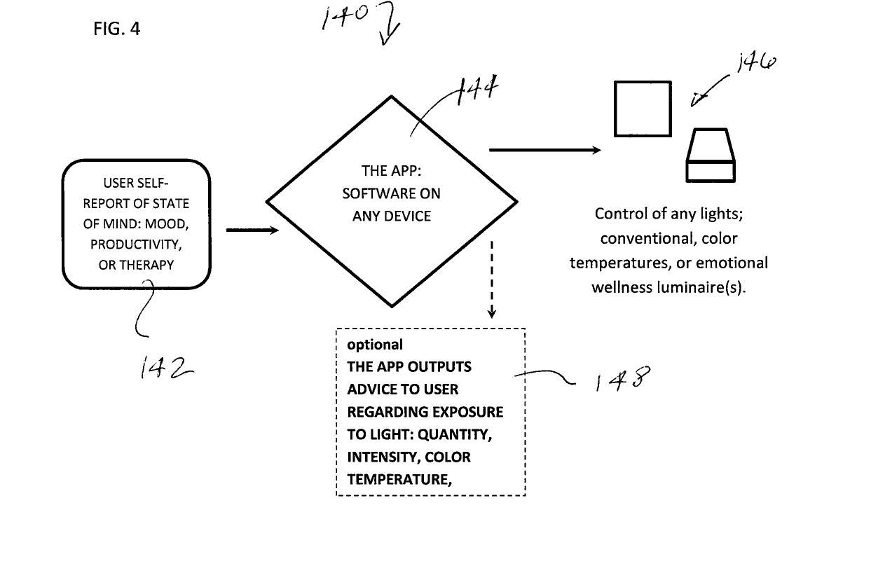

FIG. 4 depicts light exposure regulating system 140 comprising software 144 operation on memory and a processor, with the software configured to receive user self-reported data (e.g., data comprising a self-rating of emotional wellness) and/or user tracking data with the software accessing the memory to provide automated direct control of a setting of one or more luminaires 146 and/or advice 148 for a user to control a setting of a luminaire 146 with regards to color temperature or other light values. General-use and/or light therapy and/or light productivity luminaires may be controlled.

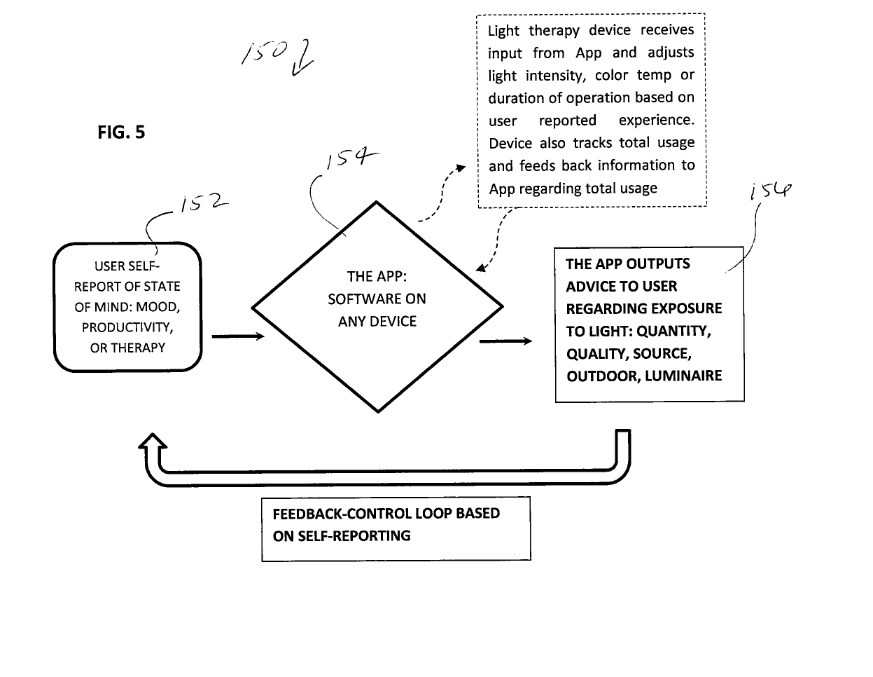

FIG. 5 depicts a light exposure regulating system 150 comprising software 154 that uses self-reported data 152 comprising a user self-rating and/or user tracking data to provide an output of advice 156 with regards to color temperature or other factors, based on user self-reported data 152, with the processor changing the advice based on ongoing user self-reported data. A feedback control loop may be established whereby user self-reporting conditions the advice given to the user. System 150 may further control luminaires directly or indirectly.

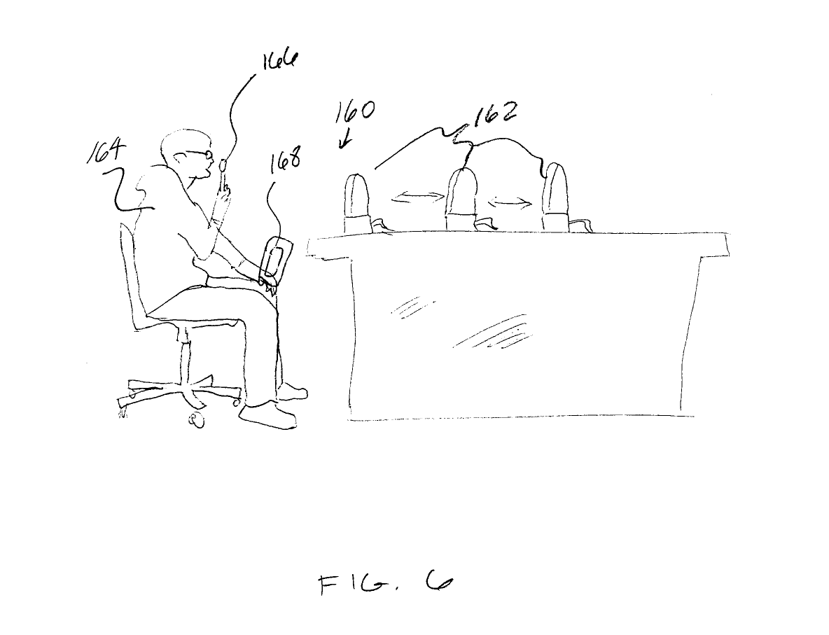

FIG. 6 depicts system 160 with a portable light therapy luminaire 162 being positioned at various distances from user 164, who holds sensor 166 that senses one or more light values, e.g., intensity and color temperature. Mobile computing device 168 is wirelessly in communication with sensor 166 to capture the values to memory. Software on device 168 provides directions to user 164 for a calibration process and/or determining the user's comfort level for various intensity/color temperature combinations. This data is used to directly or indirectly control luminaire 162. Direct control may be exercised by the software to communicate with luminaire 162 to adjust its light value settings. Indirect control may be provided in the form of instructions to the user, who then adjusts settings of luminaire 162.

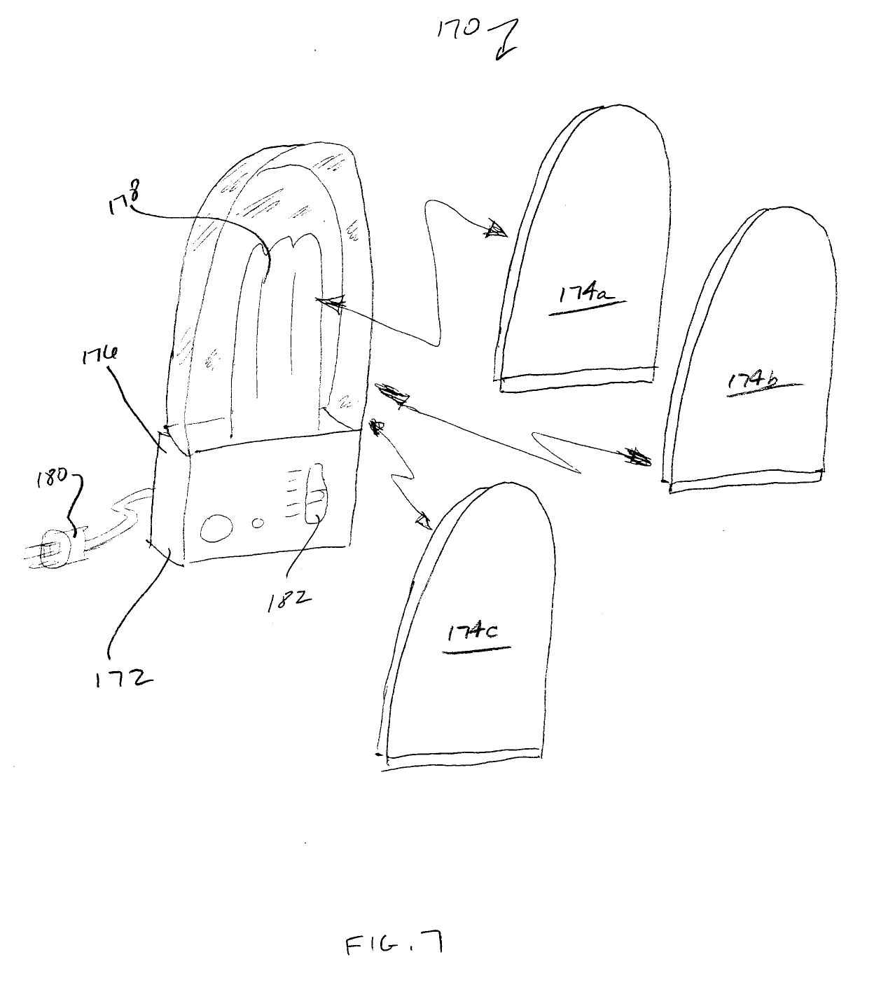

FIG. 7 depicts system 170 having portable luminaire 172 and a set of lenses 174a, 174b, and 174c. Luminaire 172 has housing 176, light source 178, electrical plug 180, and various controls, including slider 182 for selecting an intensity. Lenses 174a, 174b, 174c, each fit on housing 176 such that all of the light from source 178 must pass through the lens that is on the housing in order to be received by a user when luminaire 172 is used as designed and intended. The lenses change the color temperature of the light that is emitted from the luminaire. The depicted embodiment provides for only one lens to be mounted at a time. Alternative embodiments provide for one or more lenses to be mounted simultaneously, so that one lens may be used or a plurality of lenses may be placed in the path of the light.

The various features of these embodiments are detailed below; the features may be mixed-and-matched together with the limitation that a functional device must be made. Further embodiments are also provided, as well as variations of the above-described embodiments.

### Luminaires: General Purpose and Light Therapy

The term luminaire (or light fixture or light fitting), as commonly used and as used herein, refers to an electrical device used to create artificial light by use of an electric light source, and includes a complete lighting unit, comprising one or more light sources (bulbs or tubes or other light sources that emit light), along with a socket or the like, and a connection of the light source to a power source. It includes, when present, a diffuser or reflector that helps direct and distribute the light. Fluorescent light fixtures often have lenses or louvers. Luminaires include both portable, and permanent fixtures, for instance ceiling- or wall-mounted fixtures. Fixtures require an electrical connection to a power source; permanent light fixtures may be directly wired, and moveable (portable) luminaires have a plug. Light fixtures may also have other features, such as an aperture (with or without a lens), an outer shell (the housing) for lamp alignment and protection, and electrical ballast or a power supply. Portable light fixtures are often called “lamps”, for instance a table lamp or desk lamp, although the term lamp is properly used to refer to the actual light source. Herein, the term light source is used to refer to the object that generates the light, for instance a light bulb, a light diode, a light-emitting diode (LED), or a fluorescent tube. Sometimes the light fixture can be conveniently characterized in terms of its light source, for instance, a fluorescent light fixture or an LED light fixture.

A variety of light sources may be used, for instance, fluorescent, incandescent, halogen, light diode, and light-emitting diode (LED). A fluorescent light source (for example, a fluorescent tube) is a gas-discharge lamp that uses electricity to excite mercury vapor. The excited mercury atoms produce short-wave ultraviolet light that then causes a phosphor to fluoresce, producing visible light. Alternative sources are incandescent bulbs, although the range of color temperatures available with conventional bulbs is limited. A halogen light can be useful. These are also known as a tungsten halogen lamps or quartz iodine lamps, and are incandescent lights that have a small amount of a halogen such as iodine or bromine added. The light-emitting diode (LED) or a light diode light source can also be useful. Options include combining red, green, and blue LEDs or diodes to make a desired color of light, including a white light. Other colored light sources may alternatively be combined to make various colored or white light.

White light can be useful. There are a variety of light sources known that can produce a white light or a light that can be filtered to a white light. Lens sets can be made to change a color temperature with these light sources. Alternatively, the light source can, in some instances, be changed to alter its color temperature. For instance, when red, green, and blue lights are combined, the mix of colors can be adjusted to change a color temperature, but only if the lights and control systems are configured with this object in mind. In many cases, conventional mixed-systems (red, green, and blue lights, etc.) are not adaptable to change a color temperature of a white light. White light is one choice of many colors that are available for a luminaire. White light may be provided with a plurality of bulbs or a plurality of light emitting diodes. Embodiments may include a plurality of light sources that have different color temperatures, with light from these sources being mixed, and adjusted, to provide a desired color temperature; further, all of the sources may be white light sources.

Certain embodiments are directed to white light provided without mixing light sources that have different colors. Light may be provided from the luminaire by one or more light sources that are white and are free of non-white sources. In contrast, a lamp that uses a source that relies on a combination of, e.g., red, green, and blue lights is mixing colors of light sources. Similarly, certain embodiments are directed to white light provided from a single bulb, a plurality of light sources that each have the same color temperature, a plurality of light sources that collectively provide a single color temperature without mixing different color temperatures, or is provided from one or more sources without mixing light sources that have different color temperatures. A plurality of light sources may be used that are matched to each other, or a unitary white light source may be used; such light sources are not based on a mix of color temperatures. In the context of certain embodiments, such sources have advantages, such as better control of color temperature as intensity is changed.

### Lenses for Luminaires

The term lens, as used herein in the context of a luminaire, refers to a device used to alter light as it passes through the lens and exits the luminaire. Light passes through the lens so the light can be used for its intended purpose. Lenses are familiar in everyday use. For instance, diffusers that overlay an overheard fluorescent luminaire are lenses. And many desk lamps have lenses. On the other hand, a light fixture with only a naked bulb does not have a lens.

There are some references that refer to a “lens” as including a reflector portion of a luminaire, but the term lens as used herein does not refer to, or include, the reflector. A reflector is located inside a luminaire and redirects light from inside the luminaire. The reflector is reflecting light and is not passing the light, even if there is some subportion of the reflector that passes light, such as the film on a mirror.

A lens may be effectively non-blocking or it may block a portion of light that impinges on the lens. For instance, most diffusers pass effectively all of the light they receive. Some lenses are filters that block a portion of the light by absorbing it, usually in some spectral range. Some lenses are semi-reflective, meaning they reflect some of the impinging light but pass other portions of it. Neutral density filters pass substantially all wavelengths of impinging light but block a quantity of the light. Bandpass filters pass light within certain wavelengths and block the other portions, either by absorption or reflection.

Lens, in the context of a luminaire, is a term that encompasses both total lenses and partial lenses. A total lens passes all of the light that is emitted by a luminaire; light does not leave the luminaire unless it has passed through the total lens. It must be acknowledged that luminaires are not conventionally made to be light-tight so that some light escapes a luminaire even if a total lens is employed, e.g., through vents in a back of the luminaire. Nonetheless the luminaire, when used as intended, requires effectively all light to be passed through the total lens. A partial lens is designed to allow at least some light to escape the luminaire without passing through it.

A lens alters light that passes through it. Alteration may include, for example, diffusing, scattering, filtering, changing wavelengths, changing color, or changing a color temperature. In contrast, a wire cage for protecting a light does not substantially alter a light even if some small portion is reflected off the cage. Lenses may also be made that change an intensity of light without changing, or with little change to, a color temperature of a light.

It is significant that lenses may be made that change a color temperature of the light that is emitted from the luminaire without essentially changing an intensity of the light. In this context, the term “essentially” means that the lens decreases the photopic intensity of light passing therethrough by no more than about 15%. In general, the lenses may be designed to change a color temperature without decreasing intensity more than about 5%, about 10%, about 15%, or about 20%; artisans will immediately appreciate that all ranges and values within this range are contemplated and supported, e.g., less than 10% or no more than 12%. The color temperature change for a lens relative to the light source may be from about 1 to about 18,000 kelvins; artisans will immediately appreciate that all ranges and values within this range are contemplated and supported. In general, the lenses decrease the color temperature of the light source by at least about 500 kelvins and provide color temperatures that are at least about 500 kelvins different from each other: for example, the set may have lenses that cause a color temperature decrease of 500, 1000, and 1500 kelvins each. Accordingly, the lenses may also cause a color temperature decrease of 1000, 2000, and 3000 kelvins. Or 1000, 1800, and 3500 kelvins, and so forth; artisans will immediately appreciate that all ranges and values within these various ranges are contemplated and supported. Moreover, the range of intensity decreases (5% to 20%) and values for color temperature drops of various lenses (1 to 18,000 kelvins) may be mixed-and matched; artisans will immediately appreciate that all ranges and values within this range are contemplated and supported, e.g., a 5% or a 10% intensity drop for a 1000 or 6500 kelvins lens.

### Light Therapies, Light Therapy Luminaires, and Ambient Lighting

Environmental light is a primary stimulus to control circadian rhythms, seasonal cycles, and neuroendocrine responses. Many studies have tested the use of light for treating disorders such as Seasonal Affective Disorder (SAD), depression, sleep disorders, hormonal regulation, and eating disorders. In general, a response to light relates to its intensity, wavelength and dosage, with dosage being the product of intensity and time.

Light therapy treatment can restore circadian rhythmicity to effectively treat affective disorders and insomnia, and to increase sleep efficiency. Light therapy for SAD is generally well tolerated, with most patients experiencing clinical improvement within one to two weeks after the start of treatment. To avoid relapse, light therapy should continue through the end of the winter season until spontaneous remission of symptoms in the spring or summer. Kurlansik et al., Seasonal affective disorder, Am Fam Physician. 2012 Dec. 1; 86(11):1037-41. Light therapy is effective in treating seasonal affective disorder (SAD) and non-seasonal depression in adults, with effect sizes equivalent or superior to psychopharmacologic treatment. R. N. Golden, B. N. Gaynes, R. D. Ekstrom et al., “The efficacy of light therapy in the treatment of mood disorders: a review and meta-analysis of the evidence,” American Journal of Psychiatry, vol. 162, no. 4, pp. 656-662, 2005.

Suitable light intensities for these various conditions vary. A light therapy luminaire is a luminaire purpose-built for light therapy that provides a light intensity that falls within a range from about 2000 lux to about 30,000 lux at a range from about 6 inches to about 48 inches from the device. These intensities at this range are generally suitable for light therapy. A lux is the SI unit of illuminance and luminous emittance, measuring luminous flux per unit area. It is equal to one lumen per square meter. Conventional wisdom teaches an intensity of about 10,000 lux for SAD but applicants have determined that that the pioneering studies that established this value did not govern and measure the actual intensity with accuracy. Measurement of light intensities requires proper equipment and controlled conditions that were not available or not known to the clinicians. Specifically, depending on the ratio of various light receptors in the eye, individuals may respond differently to different color temperatures of light therapy, impacting the total perceived brightness and biological response based on the ratio of photopic to scotopic light. Many researchers, without knowledge of the impact of scotopic light on the eye and resulting circadian system, relied solely on photopic measurements during the research such that their data did not fully capture the total amount of light as perceived by the eye and its resulting impact on the patient.

Applicants have determined that many persons do not require and/or will not comfortably tolerate an intensity of 10,000 lux. Artisans will immediately appreciate that all ranges and values between the explicitly stated limits are contemplated for the light therapy devices described herein; e.g., from about 1000 lux to about 8000 lux or 5000 lux at a range of 1 inch to 48 inches and all values in between.

Ambient lighting need only be 500 Lux at the user to be effective for general purposes. It must be appreciated that a light therapy luminaire is not a general purpose workplace light fixture, a task lighting lamp, a medical laser, a medical diode laser, stage or theatre lights, a heat lamp, a lamp customized for industrial applications, or the like. Light therapy luminaires provide light within certain ranges and have a bright, intense appearance as compared to conventional workplace lighting fixtures. An overhead workplace light that provided the intensity of therapeutic light at the user's normal location in a room would be so bright as to be painful and/or damaging to the eye to look at, and would be unsuitable from various design standpoints, such as heat generation, energy consumption and government compliance.

### Measuring Light

Luminous flux (measured in lumens) is a measure of the total amount of visible light present. Light intensity can be characterized and measured in different ways: radiant intensity (W/steradian), luminous intensity (lumens/steradian), and irradiance (W/m²). The term light intensity, as used herein, also refers to values of lux, which is actually a measure of light intensity per area: one lux is equal to one lumen per square meter. The term lux relates to photopic intensity, meaning it accounts for how the eye perceives the light. The term retinal dose refers to the dose received at the retina. A lumen is the traditional measure of light intensity. The definition of the lumen is based on the response of the cone cells to light. The eye has three primary light-sensing cells in the retina, known as photoreceptors, called rods, cones and melanopsin. Cones process visual information under bright light levels. Rods mediate vision used in darkness and control the amount of light permitted to enter the eye through the pupil (scotopic conditions). The term dose refers to the product of time and intensity.

In the past, lighting manufacturers used light meters to determine lumen output, or luminous efficacy. But these devices were based on the decades-old assumption that the cones generally mediate vision and that the light sensitive rods were relevant only in low-light, or nighttime, conditions. The role of melanopsin, discovered in 2002, was not even known. And the traditional assumptions about the light source were similarly based on the lumen. Measurements in lumens can still be useful but there are further factors that can be important when assessing light output. There are additional receptors and other biological factors besides rods and cones. For instance, the ratio of scotopic luminance versus photopic luminance in a light is called the S/P ratio. The S/P ratio affects the perceived brightness intensity of light. In practice, a light source can be made to appear brighter than its actual lumens. In fact, feedback inputs from rod cells control pupil size. If the rod cells can be stimulated to open the pupil, then the dose of light received in the eye can be increased without increasing the brightness (lux) of the source. Controlling the color temperature in light therapy can provide this effect. A light source with a high S/P ratio will appear brighter than the same light source with a relatively lower S/P ratio. There are other more subtle effects, such as a decrease in light scattering that reduces eye strain. All of these factors are harnessed in embodiments of the invention that manipulate light sources to provide an intended color quality, color temperature and options to control luminaires for a desired light output. Table 1 provides correlations between color temperature and S/P ratios.

TABLE 1

| Color | |
|---|---|
| Temperature | S/P Ratio |
| 7,500K | 2.4-2.2 |
| 6,500K | 2.2-2.0 |
| 5,500K | 2.0-1.8 |
| 4,500K | 1.8-1.6 |
| 4,000K | 1.6-1.5 |

### Control of Luminaires

Embodiments include methods that involve controlling luminaires. These include light-therapy luminaires and, in some cases general purpose luminaires or other specialized luminaires. In general, a setting of a luminaire may be controlled. The term setting is broad and includes, for example, one or more of an intensity, a color, or a color temperature. There may be a variety of luminaires and settings to choose from, for instance luminaires with various color temperatures, fluorescent luminaires, and portable luminaires. A processor may use various data to control a luminaire. Further, the luminaire may have various features that can be controlled.

Such features may include, for instance, power, variable power controllers, features that adjust lamp intensity, a dimmer, a filter, a lens, a color lens, a color temperature lens, a ballast for a luminaire, one of a plurality of lamps (LED, diode, incandescent, and so forth) in a luminaire chosen independently or in combination, a lamp driver, louvres, reflectors, and lenses. Digital ballasts can be used to control a luminaire, for instance, a fluorescent luminaire. A lamp driver may be an LED lamp driver; these may be used, for instance, to change a light color, a light color temperature, an LED intensity, or a combination thereof.

Lenses, including filters, may be dynamically changeable to control the lens properties. Dynamically changeable lenses, filters, films, coatings, materials, glass, glazing, and so forth, are devices that controllably change light-altering or light-transmission properties under the application of voltage, light or heat. Such lenses or filters, when activated, change from transparent to translucent, blocking selected, or all, wavelengths of light. These technologies include electrochromic, photochromic, thermochromic, suspended particle, micro-blind and liquid crystal devices.

Mechanical controls may be used to control the luminaire and its various features. For instance, the lamp, luminaire and/or its lens may be mechanically moved. Or reflectors or louvres may be moved to block, alter, or redirect light.

Accordingly, the luminaires and the various features may be controlled to have a variety of effects. For example, an effect may be one or more of intensity, color temperature, color, and direction of light. As described in more detail elsewhere herein, a timing and/or a duration of such effects may be controlled, and the control may be in response to various data and algorithms.

### Color Temperature and Color

Color is a familiar concept. Various color models have been developed to describe colors, including theories that describe all colors as a specific hue (red, orange, yellow etc.) and value. The hue refers to the color spectrum, with the visible spectrum being from about 390 to about 700 nm. The value refers to the lightness or darkness of a color. Color temperature is a different concept that refers to light in terms of the light emitted from a black body. Color temperatures from about 2,000 to about 10,000 kelvins are referred to as white light herein. The light, before or after filtering, may be further limited to a narrower range such as from about 3,000 kelvins to about 8,000 kelvins, with this range being an alternative white light range. The white light depending on its color temperature, may be variously described as being, hot, cold, as having a bluish tinge, or in various other terms but it is, nonetheless, recognized as white light both by common use in the industry and government regulation. Artisans will immediately understand that all ranges and values between the explicitly stated values of 2,000 and 10,000 are contemplated. The term “color temperature” as used herein means the color temperature of a black body as well as a correlated color temperature that refers to light from non-incandescent and non-black body that is matched to the color temperature scale. The color temperature of a 60 W incandescent bulb is about 2700 kelvins, for a 100 W incandescent bulb is about 2900 kelvins, for a halogen bulb is about 3000 kelvins, and daylight is about 4900 kelvins. Further embodiments are directed to lights with color temperatures from 1500 to 10,000 kelvins; embodiments described herein for white light may be performed using light color temperatures from 1500 to 10,000 kelvins. Various systems and processes for controlling light variables such as color temperature are described herein. Color temperatures may be adjusted continuously or in increments. For instance, a luminaire may be adjustable to provide a plurality of color temperatures that are different from each other by an incremental amount, e.g., 100, 200, 250, 500, 700, 800, 1000, 1500, 2000, 5000, 7000, 10,000 kelvins. Thus a luminaire with a setting of 2000 and 2500 kelvins would have a color temperature adjustable by an increment of 500 kelvins. A system with a plurality of lenses that reduce color temperature by 500, 1000, and 1500 kelvins, respectively, has incremental adjustments of 500 and 1000 kelvins.

On the other hand, a system continuously adjustable across a range would not be adjustable incrementally in that range. Accordingly, embodiments herein may be provided using a luminaire and/or light source and/or lenses that: is/are not continuously adjustable with respect to color temperature. Further, the luminaire and/or light source and/or lenses may be: not continuously adjustable with respect to color temperature and intensity. Thus, for example, a luminaire may have a light source that is continuously adjustable but lenses that are not. Or the lenses may be continuously adjustable for a light source that is not continuously adjustable.

### Wearable Light Sensing Device

The wearable light sensing device measures exposure to light and/or light intensity, and preferably also measures light wavelength information. Time information is captured with the data, either real-time, time over a period, or even by the day; the sensor and/or memory can provide time indicia. The sensor, in general, generates data useful for understanding a user's total exposure to light quantity and light quality. There may be one or more sensors, for instance with various sensors in one or more devices wearable by the user, with the devices measuring different light qualities and/or providing redundancies.

A wearable (on clothing or skin) light sensing device comprises a light sensor and generates data regarding what the sensor detects. The wearable light sensing device may be lightweight, with a weight of less than 16 ounces; artisans will immediately appreciate that all ranges and values within this range are contemplated and supported, e.g., less than 1, 2, 6, 8, or 12 ounces, or from 0.1 to 8 ounces. The wearable light sensing device may alternatively or additionally be small, with a footprint of no more than 15 square inches; artisans will immediately appreciate that all ranges and values within this range are contemplated and supported, e.g., less than 1, 2, 6, 8, or 12 square inches or from 0.1 to 8 square inches. The term footprint refers to the surface area projected onto a flat surface (beneath the device) when the device is positioned according to its intended use on a user. A subcategory of the wearable device is a placeable device for placement on equipment or accessories—instead of being worn, the device is associated with the user; accordingly, a sensor could be on the person or near the person. There may be multiple sensors placed in areas where a person spends most of their day.

An embodiment uses a cellphone or the like to detect a presence of a sensor and collect the information when it is nearby, e.g., by BLUETOOTH or other protocol. Thus a user might have a sensor in one or more of various locations, e.g., the workplace, the home, the car, various rooms in the home. A sensor could be in the lights or a light therapy device.

The intended use will vary according to the design but wearable or placeable sensors are generally to be positioned where they can sense the light around the user—so they are worn on the clothes or accessory such as a hat, purse, arm or wrist band, shoe, and so forth. The sensor may also be placed on equipment such as a cellphone, camera or helmet, bicycle, etc. The device can be permanently incorporated into some other object, e.g., a hat or clothing, and the footprint would relate to the device and not the entire object that receives it. For instance, it could be in, or fastened on, clothing, a clothing accessory, a watch, jewelry. The fastening could be direct or indirect, for instance, by a ring or a chain. Attachment directly on skin, or as an adhesive patch is possible. The sensor could be disposable, for disposal after a time from 0.5 days to a year; artisans will immediately appreciate that all ranges and values within this range are contemplated and supported, e.g., daily use, weekly use, bi-weekly, monthly.

A variety of sensors for the wearable light sensing device or other light-sensing devices are available. Examples of sensors are as follows. Active-pixel sensors (APSs) are image sensors. Usually made in a CMOS process, and also known as CMOS image sensors, APSs are commonly used in cell phone cameras, web cameras, and some DSLRs. Charge-coupled devices (CCD), which are used to record images in astronomy, digital photography, and digital cinematography. Chemical detectors, such as photographic plates, in which a silver halide molecule is split into an atom of metallic silver and a halogen atom. The photographic developer causes adjacent molecules to split similarly. LEDs which are reverse-biased to act as photodiodes. See LEDs as Photodiode Light Sensors. Optical detectors, which are mostly quantum devices in which an individual photon produces a discrete effect. Optical detectors that are effectively thermometers, responding purely to the heating effect of the incoming radiation, such as bolometers, pyroelectric detectors, Golay cells, thermocouples and thermistors, but the latter two are much less sensitive. Photoresistors or Light Dependent Resistors (LDR) which change resistance according to light intensity. Normally the resistance of LDRs decreases with increasing intensity of light falling on it. Photovoltaic cells or solar cells which produce a voltage and supply an electric current when illuminated. Photodiodes which can operate in photovoltaic mode or photoconductive mode. Photomultiplier tubes containing a photocathode which emits electrons when illuminated, the electrons are then amplified by a chain of dynodes. Phototubes containing a photocathode which emits electrons when illuminated, such that the tube conducts a current proportional to the light intensity. Phototransistors, which act like amplifying photodiodes. Quantum dot photoconductors or photodiodes, which can handle wavelengths in the visible and infrared spectral regions.

As is evident, the sensors may be tuned to sense light presence, light quantity, and light quality. The quality of light refers to characteristics such a spectrum or color temperature, e.g., visible spectrum, range of wavelengths, or kelvins. The type of light source is a quality of light, for instance, daylight, fluorescent light, and incandescent light. The sensor may incorporate features to alter its native sensing condition so that it sense what is intended. For instance, the sensor may incorporate a filter that passes only desired wavelengths: an example would be a CCD with a red-green-blue sensor array that has a filter that only passes red color. Similarly sensors can be equipped to distinguish daylight from interior light. Alternatively, the sensor can report data as-received and a processor can interpret the data as may be helpful. The quantity of light refers to a total flux of light: for instance, an intense light has a higher flux than a less intense light, and a total quantity is the product of the flux over time. A dose refers to a quantity of light received at a location; often the dose at the sensor can be attributed to the user or used as a proxy for the actual dose of the user.

The sensor passes sensor data to a processor, either directly and/or through memory. Time data can be associated with the data at the sensor, at the processor, or at the memory. It is often helpful to capture time data with the sensor but it is not necessary in all cases. For instance, a sensor can record a total exposure or total dose and a processor that interrogates the sensor can record the time: for instance, the sensor data can be downloaded daily and the time captured at the download processor. Or the data can go to a processor that attributes a time to the data, which is then passed to a memory. There are many ways to pass data, for example, wireless protocols, storage-and-download, direct connection.

### Processors and Memory

The term processor is used broadly. It includes a computer chip or integrated circuit as well as a larger computing device that contains the same. It includes personal computing devices, cellular telephones, smartphones, mobile telephones, tablets, ANDROID and MAC devices, desktop computers and the like. The processor may include memory or the memory may be provided separately.

The processor and/or memory may be part of a sensing device, in a luminaire, in a custom purpose-built device, or at a remote site accessed by internet or other remote communications.

Memory refers to the physical devices used to store programs (sequences of instructions) or data (e.g. program state information) on a temporary or permanent basis for use by a processor or a digital electronic device. Memory may be, for instance, addressable semiconductor memory, i.e. integrated circuits consisting of silicon-based transistors, used for example as primary memory but also other purposes in computers and other digital electronic devices. The term memory is used broadly and encompasses volatile and non-volatile memory examples of non-volatile memory are flash memory (sometimes used as secondary, sometimes primary computer memory) and ROM/PROM/EPROM/EEPROM memory (used for firmware such as boot programs). Examples of volatile memory are primary memory (e.g., dynamic RAM, DRAM), and fast CPU cache memory (typically static RAM, SRAM, which is fast but energy-consuming and offer lower memory capacity per area unit than DRAM). RAM is random access memory, SRAM is static RAM, DRAM is dynamic RAM, ROM is read-only-memory, PROM is programmable read-only memory, EPROM is erasable programmable read only memory, EEPROM is Electrically Erasable Programmable Read-Only Memory.

Information from sensors, processors, and memory may be of any type, for instance, over wire or wireless, of with mediation from standard software, e.g., an “app” on a cellular telephone or other device. Many such processes are known. Wireless processes, for instance, may use radio or various fixed, mobile, and portable applications, including two-way radios, cellular telephones, personal digital assistants (PDAs), and wireless networking. Wireless networking is well known, and examples of wireless networks include cell phone networks and Wi-Fi local networks. Various protocols are known, e.g., Bluetooth (BLE), Zigbee, 802.11.

### Data Inputs and Outputs; Advice and Control of Devices

Data is collected by sensing and from users. User data may include user self-reporting to provide both diagnostic data and outcome data. The data is processed to generate outputs. The outputs may include control of lighting fixtures and/or advice. The advice may include recommendations for controlling the user's exposure to light, such as choice of light sources, light therapy lights, light color temperature, and intensity. Feedback loops can be used to account for outcomes and generate ongoing improvements in the advice-outputs.

Luminaires may be controlled with various techniques. The system generates a signal that is sent to the luminaire. A signal traveling by wire could control a luminaire directly, by changing a setting. Or signals, wired or wirelessly, can be received a processed by a circuit or a processor that translates to signal into a control signal that actuates a change in the luminaire.

Data may include user-provided data, for example self-reported data or user tracking data. Self-reported data may include, e.g., the user's reporting of activities, light exposure, or emotional wellness. Activities reported may be a general indication of an activity level and/or specific reports of time, type, location, and intensity of activities, for example, sedentary, walking, exercise, and indoor or outdoor. Light exposure might be, e.g., quantity, quality, light sources (indoor/outdoor, fluorescent, LED, etc.), or light intensity. Emotional wellness may be, e.g., a general indication of, or a rating on a scale of, well-being (Sadness/Happiness), anxiety, negative feelings, depressive moods, productivity, mental alertness, ability to focus, distractibility, irritability, or mindfulness. User tracking data may be, provided by grant of access from a user to, for instance: activity-tracking bands, third party health trackers, cell telephones (for instance, electronic calendars or Global positioning satellite locations), or organizational software (for instance, counts of incoming/outgoing emails, calendar data, total computer use, telephone use, calculation of total stress load based on such factor(s)).

Data may include public user-data, for instance, local weather conditions or cloud-based resources. Data may include sensor data, for instance lights sensors that are wearable or not-wearable, e.g., fixed, portable without being worn, or desktop. Fixed sensors may be in, e.g., a home, business, or automobile. Sensor data may be collected from third-party devices, e.g., noise sensors, fitness sensors, and GPS.

Data may be aggregated data collected from many users, with the experience of the many being a baseline for use with individuals. The aggregated data may be filtered by use by criteria, for instance, comparable users' experiences as selected by age, gender, fitness, geographical location, income. Data may be taken from third party data aggregations.

Data may be collected from a light sensing device. The data may comprise light quantity and/or light quality. The data may include time exposed to daylight, daylight dosage, fluorescent light exposure/dosage, indoor light exposure/dosage, therapeutic light exposure/dosage, or color temperatures. The data may include a measurement of one or more of: (i) light wavelength, light spectrum, light intensity, light intensity at a wavelength or certain range of wavelengths, outdoor light, indoor light, (ii) dosage of, or exposure to, one or more of: light wavelength, light spectrum, light intensity, light intensity at a wavelength or certain range of wavelengths, light color temperature, outdoor light, indoor light. Any of these various measurements may be related to time or a period of time or predetermined calendar times; for instance: the time of day and/or date may be captured, or recorded per minute, per hour, per day, per week.

Data may be user-provided data. User-provided data is data that the user provides directly by self-reporting or indirectly by granting access to data sources besides light sensing devices. The user-provided data may comprise a self-rating of emotional wellness and/or physical wellness and/or user tracking data. The emotional wellness may comprise one or more of: well-being, anxiety, negative feelings, productivity, mental alertness, ability to focus, distractibility, irritability, feelings of happiness, feelings of sadness, and mindfulness. The user tracking data may comprise one or more of: a calendar event, quantity of email usage, quantity of computer usage, geographical location, travel information, changes in time zones, and estimates of sleep based on user activity, user environment, presence at a workplace, the user's exposure to noise, real-time user data, and presence at a residence. User-tracking data may be captured by an automated process, for instance by access to a user's mobile computing devices to obtain GPS data, calendar appointment data, local weather at the user's site, and the like. User-tracking data refers to data collected about the user's personal experiences in time.

Self-rating data can be used to measure outcomes of the advice provided to the user. Over time, it may be that some advice is better than other advice for a particular user. For instance, self-rating of outcomes may show that a certain amount of daylight or dose of therapeutic light is preferable, and the advice can be shifted to meet the desired target. A feedback-control loop may be created to continuously optimize a user's experience.

Outputs based on processed data may include reports, alerts, advice, control of devices, and requests for further data. Reports, for instance, may be alerts or status regarding frequency of use of a light therapy or other device, a stated emotional/physical wellness status, a notice of abrupt changes in use patterns, alerts or reminders for use, and messaging regarding maintenance, e.g., change bulb/lens/sensors/ballasts/drivers/rechargeable batteries. Other outputs may be a light dose, quantity, quality, or sources of light. Reports may be made on tracked data or on historical reports, for instance status for the same time last year or for comparable circumstance.

Outputs may include advice, meaning prescriptive information for a user. Advice may be, for instance, a suggested treatment and protocol, suggested activities that are available based on a user's location or geography, or direction for current mood and disposition (e.g., here is how you fixed it last time.) Outputs may include suggested actions to achieve a targeted goal for light quantity or quality.

Outputs may be in the form of direct automated control of devices. For instance, an output may be an electronic adjustment of a luminaire settings, e.g., duration, intensity, light quality (color temperature etc.). Or other devices may be controlled. An output may be in a form of data collection, for instance, asking a user for information, collecting data from sensors and other devices, or seeking data as helpful from the web or a computing cloud.

The outputs of the processing may include reports and/or “advice”. Advice is a broad term that includes specific recommendations or guidance based on processed user data. A report is the result of, and communication of, processed data. For instance, a report might inform a user of a dose of light quality or quantity. The advice may be directed to, for example, increasing or decreasing: (1) a duration of light exposure (2) a wavelength/range of light (3) a light intensity (4) dose from a particular source (e.g., a HAPPYLIGHT, or a natural light from the outdoors) (5) light color temperature, (6) color temperature, or (7) a combination thereof.

The outputs of the processing may include a control of devices, e.g., luminaires. For instance, if the processed data indicates a need for exposure to light, or a certain kind of light, the processor may adjust a setting (low, medium, high, dim, on, off, color, color temperature) of a luminaire. There may be a variety of luminaires and setting to choose from, for instance therapeutic luminaires with various color temperatures, fluorescent luminaires, and portable luminaires. The processor may thus use the data to control the settings on a luminaire to increase or limit a time of exposure to the luminaire for a user that wears the light sensing device.

### Data Driven Methods and Systems for Control of Color Temperature

Certain embodiments of the invention are directed to products and methods for data-driven control of factors that include color temperature and/or recommendations for control of factors that include color temperature. A user provides data regarding exposure to light directly or indirectly to a software processor that assists the user in managing factors that include color temperature exposure. A user may, for instance, estimate these factors and directly enter them into a software program. The term directly refers to user-entered data that is not automatically collected; for example, entry through a keyboard, a graphical user interface, dictation, or response to queries posed by the processor software. Or data may be provided from a sensor that interfaces with the software. For example, a user may wear a wearable sensor that passes data to the software. The data may be passed automatically, for instance wirelessly when the processor detects the sensor to be within range of downloading information. Or the data may be passed by a process that requires a user's activity, for instance, docking the sensor to an interface for the processor, making a connection, transferring memory, and so forth. The data and/or input may comprise one or more of the descriptions of data and/or input described herein.

The processor that processes data can control a luminaire directly or indirectly. Directly means that the processor acts on data to adjust a luminaire without mediation by a user. Indirectly means that the processor generates an output that involves the user in making an adjustment. For instance, the user might be instructed to select a particular color temperature or to raise/lower the color temperature. Controlling the color temperature is helpful for controlling the amount and quality of light provided to the user. The control and/or output may comprise one or more of the descriptions of control and/or output described herein.

Other embodiments of the invention are directed to a system or a method for controlling a light therapy luminaire comprising a portable light therapy luminaire adjustable to change a value of light, e.g., color, intensity, or color temperature, a sensor for taking a measurement of the white light, and a software application for capturing the measurement, with a user positioning the sensor at a known distance from the luminaire, using the sensor to take the measurement, and the software application capturing the distance and the measurement, or a value derived from the measurement, into a memory. The color temperature of the white light may be changed by at least 500 kelvins without changing the light to a non-white color. A plurality of the values may be captured at one or more distances. The user, optionally with prompting or other assistance from the software, may identify what color temperatures or range of color temperatures are comfortable at one or more intensities. This information may be saved for later use in controlling the luminaire as described elsewhere herein.

Another embodiment of the invention is directed to self-monitoring light therapy methods and luminaires comprising a portable light therapy luminaire and a sensor that senses an intensity and/or color and/or color temperature of the white light. The luminaire in use, may, e.g., provide white light at an intensity of at least 2000 lux at a distance of about 6 inches from the luminaire. The light source may lose intensity during the course of use, and drop below a desired level even before its rated service life is over. And lenses on the luminaire may change color or transmissive properties over time and/or use such that the light color, intensity, or color temperature changes. The luminaire and sensor may be used with a software application for determining when a value of the light, e.g., color, color temperature, or intensity falls outside of a predetermined acceptable operating range. The determination may be made when the light has been powered-on for a length of time adequate to warm-up a light source in the lamp that provides the light.

### Processing of Data

In this example, the signal is generated based on the data collected (the inputs) and series of algorithms and comparable look up tables (dynamically populate with treatment protocols) are used to determine the adjusted dose based on input data and comparable symptoms and reactions to previous therapy or exposure to natural light. A processor runs the algorithms which determine best intensity and duration (dose) based on the combination of the user inputs and the then current state of care (CSOC) (most appropriate treatment protocol MATP). The MATP is transmitted to the light therapy device. The device has memory capable of storing the MATP and keeping track of total usage (TU). The device transmits TU data to the computer processor device (CPD). CPD updates the database and resulting look up tables with the most current use data. The Sensor transmits (wired or wirelessly) to the CPD and provides most current light exposure data. The internet connects the CPD to a master online database where the CSOC data is kept and updated either manually or ultimately automatically as the user TU and user results continue to be tallied. The CSOC is a combination of user reported data and study data (from third party clinical trials and studies). The CSOC is updated as dictated by shifts in the body of knowledge detected and entered regarding the effectiveness of SAD and pushed to the CPD. The CPD compares the various CSOC profiles with the user profile and selects a treatment protocol that most closely fits the users' then current profile. The CPD transmits the specific treatment protocol to the light therapy device. The light therapy device captures, stores and implements the therapy and records use of the device (TU), the TU is returned to the CPD, the CPD updates the specific user data, connects with the cloud and updates the CSOC and utilizes the updated information to reformulate the user specific treatment protocol (USTP). The close loop system continues dynamically updating the USTP further adjusting treatment to more closely match the users' needs and the current standard of care. The user would have preset (Preferences) that would override the automated treatment protocol ensuring the user never receives a treatment that is outside their desired dosage regardless of the systems predetermined treatment plan.

The algorithms can be used to provide initial use recommendations based on user input goals and data. These recommendations would include timing, light intensity and duration. For example, research has shown that an effective course of light therapy to treat SAD is 10,000 lux for 30 minutes upon waking. Based on data received from the user, both user input and user tracked, these initial recommendations could be modified, for example by modifying treatment duration and/or intensity based on light sensor data about the duration and intensity of light received by the user during daily activities.

Recommended light color temperature and/or intensity could be reduced if the user indicated that light was uncomfortable or by reducing duration, light intensity, light color temperature or a combination thereof if negative side effects, e.g., sleeplessness occurred. Treatment timing and duration could be modified based on travel to a new location with more or less light or a change in time zone.

For SAD, typical length of treatment is 6 to 8 weeks. After that time, a maintenance schedule could be recommended to the user once user data indicated successful abatement of SAD symptoms.

### FURTHER DESCRIPTION

1. (A) A light exposure regulating system comprising a wearable, mobile or fixed light sensing device, a memory, a processor, and a luminaire having a plurality of settings, with the memory configured to receive light sensor data from the light sensor, and the processor accessing the memory to process the light sensor data to select one of the settings of the luminaire. (FIG. 1); or (B) A light exposure regulating system comprising a wearable, mobile or fixed light sensing device, a memory, a processor, and with the memory configured to receive light sensor data from the light sensor, and the processor accessing the memory to process the light sensor data to provide advice to a user of the light sensing device. (FIG. 2) or (C) A light exposure regulating system comprising a processor that applies user-self-reported data. For instance, data comprising a self-rating of emotional or physical wellness and/or user tracking data, with the program providing an output based on the user-provided datum. The output may be advice, a report, control of a device, and so forth. (FIG. 3) or (D) A light exposure regulating system comprising a memory and a processor, with the memory configured to receive user self-reported data (e.g., data comprising a self-rating of emotional wellness) and/or user tracking data and with the processor accessing the memory to provide automated control of a setting of a luminaire and/or advice for a user to control a setting of a luminaire. General-use and/or light therapy and/or light productivity luminaires may be controlled. (FIG. 4) or (E) A light exposure regulating system comprising a processor that uses self-reported data comprising a self-rating of emotional wellness and/or user tracking data to provide an output of advice based on the user self-reported data, with the processor changing the advice based on ongoing user self-reported data. (FIG. 5); 2. The system of 1 wherein the wearable light sensing device weighs less than 8 ounces and/or has a footprint of no more than 5 square inches. 3. The system of 1 wherein the wearable light sensing device comprises a fastener for fastening to a user's clothing, or has a flat surface for resting on a flat surface such as a desk. 4. The system of 1 wherein the wearable light sensing device comprises the memory, the processor, or both. 5. The system of 1 wherein the wearable light sensing device comprises one or more of: a visible light sensor, a sensor for only a portion of visible light, a sensor only for outdoor light, a sensor only for indoor light, a light color temperature sensor, a plurality of light sensors, or a combination of the same. 6. The system of 1 wherein the wearable light sensing device comprises a filter to filter light for a light sensor. 7. The system of 1 wherein the memory is disposed in the light sensing device, the luminaire, the processor, or a combination thereof. 8. The system of 1 wherein the memory is volatile memory or non-volatile memory. 9. The system of 1 wherein the processor is disposed in the light sensing device, the luminaire, the processor, or a combination thereof. 10. The system of 1 wherein the processor is a computer chip, a personal computer, a mobile computing device, a cellular telephone, or software. 11. The system of 1 wherein the luminaire is a portable fixture, a permanent fixture, a fluorescent light fixture, an LED light fixture, or a light therapy luminaire. 12. The system of 1 wherein the plurality of settings includes at least two of the following: on, off, dim, low, medium, high. 13. The system of 1 wherein the memory is configured to receive light sensor data from the light sensor with a device or process that comprises: wireless communication, electronic communication, wired electronic communication, optical signaling, or direct input from the sensor. 14. The system of 1 wherein the memory is on the light sensing device, with the memory being accessed automatically or on command by the processor, either wirelessly or by direct connection. 15. The system of 1 wherein the light sensor data comprises one or more of: (i) a measurement of one or more of: light wavelength, light spectrum, light intensity, light intensity at a wavelength or certain range of wavelengths, outdoor light, indoor light, (ii) dosage of, or exposure to, one or more of: light wavelength, light spectrum, light intensity, light intensity at a wavelength or certain range of wavelengths, outdoor light, indoor light, (iii) one or more of (i) and/or (ii) as related to time or a period of time or predetermined calendar times. 16. The system of 1 wherein the light sensor data comprises light quantity and/or light quality. 17. The system of 1 wherein the light sensor data comprises one or more of: time exposed to daylight and daylight dosage. 18. The system of 1 wherein the processor uses the light sensor data to control the settings on the luminaire to increase or limit a time of exposure to the luminaire for a user that wears the light sensing device. 19. The system of 18 wherein the processor compares the data to a target amount of light exposure and/or light dosage. 20. The system of 19 wherein the target amount of light exposure and/or light dosage is adjusted for a user according to user-provided data. 21. The system of 18 wherein the processer user-provided data that comprises a self-rating of emotional wellness and/or user tracking data. The processor may further or alternatively use user-provided light exposure or light dose data. 22. The system of 21 wherein the emotional wellness comprises one or more of: well-being, anxiety, negative feelings, productivity, mental alertness, ability to focus, distractibility, irritability, and mindfulness. 23. The system of 21 wherein user tracking data (which is a term not including light-exposure data) comprises one or more of: a calendar event, quantity of email usage, quantity of computer usage, geographical location, travel information, changes in time zones, estimates of sleep based on user activity, user environment, presence at a workplace, the user's exposure to noise, real-time user data, and presence at a residence. 24. The system of 1 further comprising passing the data to other processors. 25. The system of 1 wherein the advice comprises one or more light-related advice selected from the group consisting of seeking more or less of: a light exposure/dose, a quality or quantity of light, sunlight, artificial light, fluorescent light, light therapy luminaire light, visible spectra light, a portion of visible spectra light, and control of a setting for one or more luminaires. 26. The system of 1 wherein the advice comprises one of more non-light-related advice selected from the group consisting of: exercise, nutrition. 27. The system of 1 wherein the advice is based on historical data collected over at least three months or at a time that is at least three months in the past. 28. The system of 1 wherein the advice is based on past user-input emotional wellness data. 29. The system of 1 wherein the advice is based on past user-input emotional wellness data that correlates to a present time of the user. For instance, based on a correlation to a prior calendar dates that correspond to a present season, e.g., spring, summer, fall, winter. For instance, based on a correlation to a prior event or metric.

30. (A) A method of regulating a light exposure of a user comprising collecting light sensor data from a wearable, mobile or fixed light sensing device associated with the user and processing the light sensor data to select a settings of a luminaire. The user wears the wearable light sensor. The processor may access a memory that is operably associated with the sensor. (FIG. 1) or (B) The embodiment of 30(A) wherein the processor further provides advice to the user of the light sensing device. (FIG. 2) or (C) A method of regulating a light exposure of a user comprising collecting user-self-reported data, processing the data with a processor, and providing an output regarding light exposure of the user based on the user-provided datum. For instance, data comprising a self-rating of emotional or physical wellness and/or user tracking data, with the program. The output may be advice, a report, control of a device, and so forth. (FIG. 3) or (D) The method of 30 (C) further comprising automated control of a setting of a luminaire and/or an output comprising advice for a user to control a setting of a luminaire. General-use and/or light therapy and/or light productivity luminaires may be controlled. A memory may be used to store the self-reported data, with the processor accessing the data for the processing (FIG. 4) or (E) A method of regulating a light exposure of a user comprising collecting self-reported data comprising a self-rating of emotional wellness and/or physical wellness and/or user tracking data to provide an output of advice based on the user self-reported data, with the processor changing the advice based on ongoing user self-reported data. (FIG. 5). 31. The method of 30 wherein the wearable light sensing device weighs less than 8 ounces and/or has a footprint of no more than 5 square inches. 32. The method of 30 wherein the wearable light sensing device comprises a fastener for fastening to a user's clothing, or has a flat surface for resting on a flat surface such as a desk. 33. The method of 30 wherein the wearable light sensing device is wearable on, or is worn by the user on, clothing, a clothing accessory, a watch, jewelry. The fastening could be direct or indirect, for instance, by a ring or a chain. 34. The method of 30 wherein the wearable light sensing device is attached directly on skin, and/or comprises an adhesive patch. 35. The method of 30 wherein the wearable light sensing device is disposable, for disposal after a time from 0.5 days to a year. 36. The method of 30 wherein the wearable light sensing device comprises the memory, the processor, or both. 37. The method of 30 wherein the wearable light sensing device comprises one or more of: a visible light sensor, a sensor for only a portion of visible light, a sensor only for outdoor light, a sensor only for indoor light, a light color temperature sensor, a plurality of light sensors, or a combination of the same. 38. The method of 30 wherein the wearable light sensing device comprises a filter to filter light for a light sensor. 39. The method of 30 wherein the memory is disposed in the light sensing device, the luminaire, the processor, or a combination thereof. 40. The method of 30 wherein the memory is volatile memory or non-volatile memory. 41. The method of 30 wherein the processor is disposed in the light sensing device, the luminaire, the processor, or a combination thereof. 42. The method of 30 wherein the processor is a computer chip, a personal computer, a mobile computing device, a cellular telephone, or software. 43. The method of 30 wherein the luminaire is a portable fixture, a permanent fixture, a fluorescent light fixture, an LED light fixture, or a light therapy luminaire. 44. The method of 30 wherein the plurality of settings includes at least two of the following: on, off, dim, low, medium, high. 45. The method of 30 wherein the memory is configured to receive light sensor data from the light sensor with a device or process that comprises: wireless communication, electronic communication, wired electronic communication, optical signaling, or direct input from the sensor. 46. The method of 30 wherein the memory is on the light sensing device, with the memory being accessed automatically or on command by the processor, either wirelessly or by direct connection. 47. The method of 30 wherein the light sensor data comprises one or more of: (i) a measurement of one or more of: light wavelength, light spectrum, light intensity, light intensity at a wavelength or certain range of wavelengths, outdoor light, indoor light, (ii) dosage of, or exposure to, one or more of: light wavelength, light spectrum, light intensity, light intensity at a wavelength or certain range of wavelengths, outdoor light, indoor light, (iii) one or more of (i) and/or (ii) as related to time or a period of time or predetermined calendar times. 48. The method of 30 wherein the light sensor data comprises light quantity and/or light quality. 49. The method of 30 wherein the light sensor data comprises one or more of: time exposed to daylight and daylight dosage. 50. The method of 30 wherein the processor uses the light sensor data to control the settings on the luminaire to increase or limit a time of exposure to the luminaire for a user that wears the light sensing device. 51. The method of 50 wherein the processor compares the data to a target amount of light exposure and/or light dosage. 52. The method of 51 wherein the target amount of light exposure and/or light dosage is adjusted for a user according to user-provided data. 53. The method of 50 wherein the processer user-provided data that comprises a self-rating of emotional wellness and/or user tracking data. The processor may further or alternatively use user-provided light exposure or light dose data. 54. The method of 53 wherein the emotional wellness comprises one or more of: well-being, anxiety, negative feelings, productivity, mental alertness, ability to focus, distractibility, irritability, and mindfulness. 55. The method of 53 wherein user tracking data (which is a term not including light-exposure data) comprises one or more of: a calendar event, quantity of email usage, quantity of computer usage, geographical location, travel information, changes in time zones, estimates of sleep based on user activity, user environment, presence at a workplace, the user's exposure to noise, real-time user data, and presence at a residence. 56. The method of 30 further comprising passing the data to other processors. 57. The method of 30 wherein the advice comprises one or more light-related advice selected from the group consisting of seeking more or less of: a light exposure/dose, a quality or quantity of light, sunlight, artificial light, fluorescent light, light therapy luminaire light, visible spectra light, a portion of visible spectra light, and control of a setting for one or more luminaires. 58. The method of 30 wherein the advice comprises one of more non-light-related advice selected from the group consisting of: exercise, nutrition. 59. The method of 30 wherein the advice is based on historical data collected over at least three months or at a time that is at least three months in the past. 60. The method of 30 wherein the advice is based on past user-input emotional wellness data. 61. The method of 30 wherein the advice is based on past user-input emotional wellness data that correlates to a present time of the user. For instance, based on a correlation to a prior calendar dates that correspond to a present season, e.g., spring, summer, fall, winter. For instance, based on a correlation to a prior event or metric.

62A. A data driven method for controlling a light therapy luminaire comprising a portable light therapy luminaire that, in use, provides white light at an intensity of at least 2000 photopic lux at a distance of about 6 inches from the luminaire, with the luminaire being adjustable to change a color temperature of the white light by at least 500 kelvins without changing the light to a non-white color, and a software application that receives light exposure data for a user to process the data to set and/or calculate a target color temperature for the luminaire for the user. 62B. A data driven system for controlling a light therapy luminaire comprising a portable luminaire that provides white light to a user at an intensity of at least 2000 photopic lux at a distance of about 6 inches to about 48 inches from the luminaire, with the luminaire being adjustable to change a color temperature of the white light by at least 500 kelvins without changing the light to a non-white color, and a software application for receiving light exposure data to set and/or calculate a target color temperature for the luminaire. 63. The method or system of 62 wherein the data comprises an amount of time that a particular user has spent within about 6 inches to about 48 inches of the lamp. 64. The method or system of 63 wherein the data further comprises a color temperature of the lamp during the amount of time. 65. The method or system of 62 wherein the data comprises a time of exposure of the user to light. 66. The method or system of 62 wherein the data comprises a time of exposure of the user to indoor light and a time of exposure of the user to outdoor light. 67. The method or system of 62 wherein the user is presented the calculated target color temperature. 68. The method or system of 67 wherein the user is presented the calculated target color temperature with a display, a computer screen, a mobile device screen, or by audio. 69. The method or system of 62 wherein the luminaire is automatically adjusted by an output from the application to provide the target color temperature. 70. The method or system of 69 wherein the luminaire comprises a wireless receiver that receives an instruction from the software to provide the target color temperature. 71. The method or system of 62 wherein the color temperature is from 2000 to 10000 kelvins. OR a light source with a color temperature within the range of 1,500 to 10,000 kelvins. 72. The method or system of 62 wherein the color temperature is adjustable in increments, with the increments being at least 500 kelvins. 73. The method or system of 62 further comprising at least one lens to alter the light, wherein the light color temperature is adjustable by placement of the lens onto the luminaire, wherein the lens changes a color temperature of the light by at least 500 kelvins without changing the color of the light to a non-white color. 74. The method or system of 73 comprising at least two of the lenses. 75. The method or system of 62 further comprising a dynamically changeable coating for adjusting the color temperature. 76. The method or system of 62 wherein the color temperature is kept within a range from 2000 to 10,000 kelvins. Or, alternatively, the light source color temperature is within 1500 to 10,000. 77. The method or system of 6262 wherein a user manually enters the data into the software application. 78. The method or system of 62 further comprising a sensor for collecting the light exposure data. 79. The method or system of 78 wherein the light exposure data further comprises manually entered data. 80. The method or system of 78 wherein the sensor is a wearable sensor. 81. The method or system of 62 wherein the software application controls the luminaire to set a color temperature. 82. The method or system of 81 wherein the software application further controls the luminaire to provide a time of exposure to the luminaire for a user that wears the light sensing device. 83. The method or system of 62 wherein the software application controls or recommends a color temperature that depends on the light exposure data. 84. The method or system of 62 comprising collecting user-outcome self-rating data over a period of time of at least one week or at least one year, with the self-rating data being used to calculate a color temperature setting. 85. The method or system of 84 wherein the self-rating data comprises emotional wellness and/or user tracking data. The software application may further or alternatively use user-provided light exposure or light dose data. 86. The method or system of 84 wherein the emotional wellness data comprises a self-rating of one or more of: well-being, anxiety, negative feelings, productivity, mental alertness, ability to focus, distractibility, irritability, and mindfulness. 87. The method or system of 62 wherein the software application collects user comfort data at a plurality of color temperature settings. 88. The method or system of 62 further comprising a sensor, wherein the software application cooperates with the sensor to collect color temperature and/or light intensity data for the luminaire to establish the actual color temperature and/or light intensity of the luminaire at a plurality of distances from the luminaire. 89. The method or system of 88 further comprising capturing user comfort data at a plurality of color temperature settings. 90. The method or system of 88 or 89 wherein the software application controls the color temperature and provides instructions to the user. 91. The method or system of 88 or 89 wherein the user adjusts the color temperature in response to prompts from the software application. 92. The method or system of any of 88-90 wherein the software application recommends an exposure time and a color temperature to the user. 93. The method or system of 88 wherein the software application controls the luminaire to execute a calibration process that involves giving direction to the user and receiving impressions from the user as to comfort for a plurality of color temperature settings. 94. The method or system of any of 1-93 wherein the white light is provided without mixing light sources that have different colors. 95. The method or system of 94 wherein the white light is provided from a single bulb. 96. The method or system of 94 wherein the white light is provided from a plurality of light sources that each have the same color temperature. 97. The method of system of 94 or 96 wherein the white light is provided with a plurality of bulbs or a plurality of light emitting diodes. 95. The method or system of any of 62-93 wherein the luminaire has a plurality of light sources that collectively provide a single color temperature without mixing different color temperatures. 96. The method or system of any of 62-93 wherein the white light is provided without mixing light sources that have different color temperatures.

97A. A self-monitoring light therapy luminaire comprising a portable light therapy luminaire that, in use, provides white light at an intensity of at least 2000 lux at a distance of about 6 inches from the luminaire, and a sensor that senses an intensity and/or color and/or color temperature of the white light. 98B. A self-monitoring light therapy luminaire comprising a portable light therapy luminaire that, in use, provides white light at an intensity of at least 2000 lux at a distance of about 6 inches from the luminaire, and a sensor for detecting an intensity and/or color and/or color temperature of the white light. 99. The method or system of 97 further comprising a software application for determining when an intensity of the light falls outside of a predetermined acceptable operating range. 100. The method or system of 97 further comprising a software application for determining when color or color temperature of the light falls outside of a predetermined acceptable operating range. 101. The method or system of any of 97-99 wherein the determination is made when the light has been powered-on for a length of time adequate to warm-up a light source in the lamp that provides the light.

102A. A method for controlling a light therapy luminaire comprising a portable light therapy luminaire that, in use, provides white light at an intensity of at least 2000 photopic lux at a distance of about 6 inches from the luminaire, with the luminaire being adjustable to change a value of the light, a sensor for taking a measurement of the white light, and a software application for capturing the measurement, with a user positioning the sensor at a known distance from the luminaire, using the sensor to take the measurement, and the software application capturing the measurement, or a value derived from the measurement, into memory. 102B. A system for controlling a light therapy luminaire comprising a portable light therapy luminaire that, in use, provides white light at an intensity of at least 2000 photopic lux at a distance of about 6 inches from the luminaire, with the luminaire being adjustable to change a value of the light, a sensor for taking a measurement of the white light, and a software application for capturing into memory the measurement or a value derived from the measurement. 103. The method or system of 102 wherein the value of the light is color, intensity, or color temperature. 104. The method or system of any of 102-103 wherein a plurality of measurements and/or values are captured. 105. The method or system of any of 102-105 wherein the sensor displays the value and the user enters the value into a software application; wherein the measurement is a value that the sensor electronically passes to the software application; or wherein the measurement is passed electronically to the software application which derives a value of the light from the measurement. 106. The method or system of any of 102-105 wherein a software application collects user comfort data at a plurality of color temperatures. 107. The method or system of any of 105-106 wherein the software application cooperates with the sensor to establish the actual color temperature and/or light intensity of the luminaire at a plurality of distances from the luminaire. 108. The method or system of any of 105-107 wherein the software application controls the color temperature and provides instructions to the user. 109. The method or system of any of 105-107 wherein the user adjusts the color temperature in response to prompts from the software application. 110. The method or system of any of 102-109 wherein the software application controls the luminaire to execute a calibration process that involves giving direction to the user and receiving impressions from the user as to comfort for a plurality of color temperature settings. 111. The method or system of any of 102-110 wherein the white light is provided without mixing light sources that have different colors.

1. A data driven system for controlling a light therapy luminaire comprising
a portable luminaire that provides white light to a user at an intensity of at least 2000 photopic lux at a distance of about 6 inches to about 48 inches from the luminaire, with the luminaire being adjustable to change a color temperature of the white light by at least 500 kelvins without changing the light to a non-white color, and
a software application for receiving light exposure data to set a target color temperature for the luminaire.

2. The system of claim 1 wherein the data comprises an amount of time that a particular user has spent within about 6 inches to about 48 inches of the lamp.

3. The system of claim 2 wherein the data further comprises a color temperature of the lamp during the amount of time.

4. The system of claim 1 wherein the data comprises a time of exposure of the user to light.

5. The system of claim 1 wherein the software application is configured to present the user with the target color temperature.

6. The system of claim 1 wherein the luminaire is automatically adjustable by the application to provide the target color temperature.

7. The system of claim 1 further comprising at least one lens to alter the light, wherein the light color temperature is adjustable by placement of the lens onto the luminaire, wherein the lens, when in place, changes a color temperature of the light by at least 500 kelvins without changing the color of the light to a non-white color.

8. The system of claim 12 comprising at least two of the lenses.

9. The system of claim 1 further comprising a dynamically changeable coating for adjusting the color temperature.

10. The system of claim 1 wherein the color temperature is within a range from at least 2500 to no more than 10,000 kelvins.

11. The system of claim 1 wherein the software application accepts the data by manual entry.

12. The system of claim 1 further comprising a sensor for collecting the light exposure data.

13. The system of claim 12 wherein the sensor is a wearable sensor.

14. The system of claim 1 wherein the software application is operably connected to the luminaire to adjust the luminaire to the target color temperature.

15. The system of claim 1 wherein the software application is programmed to control or recommend a color temperature that depends on the light exposure data.

16. The system of claim 1 wherein the data comprises user-outcome self-rating data collected over a period of time of at least one month.

17. The system of claim 1 wherein the software application is programmed to collect user comfort data at a plurality of color temperature settings.

18. The system of claim 17 wherein the software application is programmed to prompt a user to adjust the color temperature.

19. The system of claim 17 wherein the software application is programmed to control the luminaire to execute a calibration process comprising giving instructions to the user and receiving impressions from the user as to comfort for a plurality of color temperature settings.

20. The system of claim 1 wherein the white light is provided without mixing light sources that have different colors.

21. The system of claim 1 wherein the white light is provided from a plurality of light sources that each have the same color temperature.

22. A self-monitoring light therapy luminaire comprising
a portable light therapy luminaire that, in use, provides white light at an intensity of at least 2000 lux at a distance of about 6 inches from the luminaire, and
a sensor that senses an intensity and/or color and/or color temperature of the white light.

23. A system for controlling a light therapy luminaire comprising
a portable light therapy luminaire that, in use, provides white light at an intensity of at least 2000 photopic lux at a distance of about 6 inches from the luminaire, with the luminaire being adjustable to change a value of the light,
a sensor for taking a measurement of the white light, and
a software application for capturing into memory the measurement or a value derived from the measurement.
